(19) United States
(12) Patent Application Publication
(10) Pub. No.: US 2012/0020850 A1
(43) Pub. Date: Jan. 26, 2012

(54) CRYSTALLIZATION SYSTEM AND METHOD FOR PROMOTING CRYSTALLIZATION

(75) Inventor: Morten Sommer, Boston, MA (US)

(73) Assignee: MICROLYTIC APS, Roskilde (DK)

(21) Appl. No.: 13/202,952

(22) PCT Filed: Feb. 23, 2010
(86) PCT No.: PCT/DK2010/050045

§ 371 Date: Sep. 26, 2011

Related U.S. Application Data
(60) Provisional application No. 61/154,527, filed on Feb. 23, 2009.

(30) Foreign Application Priority Data
Feb. 24, 2009 (DK) PA 2009 00245

(51) Int. Cl. G01N 33/50; B23P 11/00; B01D 9/00

(52) U.S. Cl. 422/430; 422/245.1; 29/428

(57) ABSTRACT

A crystallization system includes a well plate and a cover for the well plate, at least one of the well plate and said cover includes at least a transparent window. The well plate including at least one well comprising a bottom surface including a first essentially planar bottom surface section and a second essentially planar bottom surface section in a first bottom plane and a well border wall provided by a well border edge surrounding the planar bottom surface section: The cover includes a first essentially planar top surface section in a first top plane adapted to face the first essentially planar bottom surface section. The first and the second essentially planar bottom surface sections are totally or partly separated by a liquid barrier, provided by one or more of a low tension surface barrier, a ridge and an indentation. A method of producing the crystallization system includes providing a bottom plate, a perforated plate and a cover, mounting the bottom plate to the perforated plate to provide a well plate including a plurality of wells each having a bottom surface. The system is simple and cost effective to produce and simple to handle.

### TECHNICAL FIELD

The invention relates to a crystallization system for promoting crystallization of a target molecule, such as a macromolecule for example proteins, nucleic acids and/or carbohydrates. The invention also relates to a double chamber crystallization system for promoting crystallization of a target molecule, a kit comprising a crystallization system and a test liquid as well as a method of promoting crystallization.

### BACKGROUND ART

Crystallization of molecules, such as macromolecules, is an important technique for the biochemistry art. Biochemical molecules, such as nucleic acids, proteins and carbohydrates have unpredictable crystallization structures, and often the 3D structure of the crystallized molecules plays an important role for their biological functions. To get detailed knowledge about the way a protein functions it is critical to determine the three dimensional structure of the protein, since 3D structure and function are very tightly coupled. When biological processes need to be manipulated, the 3D structure is particularly useful, which is seen in medical research. Today more that 90% of the drugs on the market are small ligands that interact with a protein. To understand this interaction and to exploit it in the creation of new and improved drugs, the 3D structure of the ligand-protein complex has to be determined.

Crystallization of molecules e.g. macromolecules, such as proteins is performed by providing a solution of the target compound, and altering the chemical environment of the dissolved target compound such that the target becomes less soluble and reverts to its solid form in crystalline form. This change in chemical environment is typically accomplished by introducing a precipitant that makes the target compound less soluble.

The prior art discloses several methods and crystallization devices for promoting crystallization of macromolecules e.g. for screening such molecules. Three main groups of methods for promoting crystallization of macromolecules from a solution thereof include a) crystallization wherein the target solution and a precipitant are brought into contact in a capillary device and the liquids are mixed solely by diffusion b) crystallization wherein the target solution and a precipitant are brought into contact or mixed together on a well—i.e. the mixing may be both physical and by diffusion and c) crystallization wherein the target solution and a precipitant are kept physically separated but in vapor communication with one another. Because of the different nature of the macromolecules the various types of crystallization methods work with a different success rate for different types of macromolecules.

For the a) type crystallization is for example used microfluidic devices such as the devices described in WO 2008 000276 and U.S. Pat. No. 6,409,832.

For the b) type crystallization is often used ordinary well plates, such as the well plate described in US 2003/0232967 and US 2008/0230386.

A well know vapor diffusion method is a method where solvent components that evaporate from a target solution containing macromolecules to be crystallized are allowed to be absorbed by a precipitant contained in the same container. This allows the protein solution to be maintained in a supersaturation state and thereby crystals are generated gradually. In order to crystallize protein by the vapor diffusion method, a hanging droplet technique or a sitting droplet technique has been provided. In the hanging droplet technique, a solvent is evaporated in a hanging state where a droplet of a target solution is deposited and kept on the lower surface of a solution holding surface. In the sitting droplet technique, a solvent is evaporated in a seating state where a droplet of a target solution is deposited and kept on the upper surface of a solution holding part. Examples of hanging droplet crystallization devices and sitting droplet crystallization devices are described for example in US20070020748, U.S. Pat. No. 6,296,673 and EP1699538.

The objective of the invention is to provide a device for promoting crystallization of a target molecule, which device is simple and inexpensive to produce, and simple to operate.

This objective has been achieved by the invention as it is defined in the claims. And as it will be explained below, the invention and embodiments of the invention exhibit further beneficial properties compared with prior art crystallization devices and methods.

### DISCLOSURE OF INVENTION

The device of the invention for promoting crystallization of target molecules has thus shown to be very economical compared with prior art devices. One reason for this is that it is inexpensive to produce and simultaneously very simple and fast and reliable in use.

The term ‘for promoting crystallization of target molecules’ includes both the formation of the first crystals (crystal germs), as well as further growing of crystals. For some tests it is desired to examine small crystals, for other tests it may be desired to allow the crystal growth to proceed until larger crystals are formed.

The crystallization system of the invention comprises a well plate and a cover for the well plate. The well plate comprises at least one well comprising a bottom surface comprising a first essentially planar bottom surface section in a first bottom plane and a well border wall provided by a well border edge surrounding said planar bottom surface section.

In one embodiment of the invention at least one of said well plates and said cover comprise at least a transparent window, and the cover comprises a first essentially planar top surface section in a first top plane adapted to face said first essentially planar bottom surface section, preferably such that said first bottom plane and said first top plane are essentially parallel and having a ‘first bottom plane-cover distance’ D₁ of from about 10 μm to about 1000 μm, when the cover is applied onto said well plate.

This embodiment provides a crystallization system which is very simple to use. In particular it will be extremely simple to examine the sample from crystallized structures and the quality thereof, because the crystallized molecules need not be harvested and simultaneously a very good and reliable examination of the crystallized molecules can be obtained even without need for removing the cover and thereby disturbing the crystallized structure of the target molecule. When using prior art well based crystallization devises, harvesting or at least removal of the cover is a prerequisite for analyzing the crystallized structure of the target molecule.

Because the ‘first bottom plane-cover distance’ D₁ in one embodiment is very small, the target solution when applied as a droplet onto the first essentially planar bottom surface section, will be at least brought into physical contact with said first essentially planar top surface section, and preferably the target solution when applied as a droplet onto the first essentially planar bottom surface section, will be at least slightly compressed by said first essentially planar top surface section. Thereby the crystallized structure of the target molecule can be visually or optically analyzed through said window either from the top when the window is in the cover or by being turned such that the first essentially planar top surface section supports the droplet of target solution and the crystallized structure of the target molecule can be visually or optically analyzed through the window in the well plate. Alternatively both the cover and the well plate comprise] a transparent window or are entirely of transparent material as described below, in which situation the inspection and analyzing of the crystallized structure of the target molecule will be even more improved.

Also it has been found that by providing the ‘first bottom plane-cover distance’ D₁ of the crystallization system sufficiently small to ensure that the target solution when applied as a droplet onto the first essentially planar bottom surface section, will be at least brought into physical contact with said first essentially planar top surface section, and preferably be at least slightly compressed, the analysis of the molecule crystal structure will be improved by reducing or completely avoiding any shadow-effects, which would for example be present if optically analyzing a crystal in a drop due to the convex shape of such drop.

The term well plate is used to denote any solid structure with at least one well shaped structure in the form of a cavity with at least one essentially planar bottom surface section. The term “essentially planar surface” is used to mean that it should have a surface section which on a macro-level is planar i.e. visually inspected the surface section should appear to be free of protrusions and craters. In the following the term “the/said planar surface” is used to denote an “essentially planar surface”.

In one embodiment the well plate comprises a bottom wall providing said first essentially planar bottom surface section, said bottom wall preferably has an essentially planar outer bottom surface opposite to said first essentially planar bottom surface section. Thereby the well can easily be placed in a stable manner on a table and furthermore it is simple to produce. The well may for example be produced from two planar plates where one of them has been provided with holes to provide the wells, where after the plates have been fixed to each other.

In one embodiment of the invention at least a section of the bottom wall of the well plate is transparent to provide at least a transparent window into said well. In one embodiment the bottom wall or at least a transparent window thereof may be mainly or entirely of a transparent material. In one embodiment the well plate and the cover are transparent.

Due to the transparency of at least a apart of at least one of the well plate and the cover in combination with the structure of the crystallization system, the crystallized molecules need not being harvested for performing an analysis of the crystal structure damages related to handling of the crystals are therefore avoided using the present system, since it can be analyzed in the in situ in the crystallization system.

The transparent material should be transparent to electromagnetic waves of at least one wavelength. In principle this one or more wavelengths to which the transparent material is transparent may be any wavelength. In a preferred embodiment the transparent material is transparent to at least one wavelength selected from Infrared light (about 700 nm to about 1000 μm), visibly light (about 400 nm to about 700 nm), UV light (about 400 nm to about 10 nm) about and X-ray light (about 10 nm to about 0.01 nm). In a preferred embodiment the transparent material is transparent to a range of wavelengths. In one embodiment of the invention the transparent material is transparent to a range of wavelengths in the short range area such as a range of wavelengths selected from the wavelength from about 0.01 nm to about 700 nm. By using short wave light for the analysis, the analysis of the crystallized structure of the target molecule can be very detailed and the degrading of the crystal due to heat generation is very small if there at all.

In one embodiment of the invention the first essentially planar bottom surface section constitutes the whole bottom surface of said well. In another embodiment the first essentially planar bottom surface section constitutes only a part of the bottom surface. In one embodiment of the invention the bottom surface may for example comprise a curved section along the well border wall. In one embodiment of the invention the bottom surface may for example comprise a ditch along the well border wall e.g. to collect superfluous liquid applied in the well for ensuring or reducing the risk that the well be overflooded.

In one embodiment of the invention the bottom surface of the well comprises a second essentially planar bottom surface section. The second essentially planar bottom surface section may in one embodiment be in said first bottom plane i.e. the first and the second essentially planar bottom surface sections are in same plane. As it will be explained below this embodiment with the first and the second essentially planar bottom surface sections provides a whole new and simple concept for providing vapor crystallization methods, where the target solution and the precipitant are applied in same level without contacting each other physically.

The first and said second essentially planar bottom surface sections may in one embodiment be totally or partly separated by a liquid barrier. The liquid barrier may in principle have any shape, but in order not to take up too much space it is desired that the liquid barrier preferably be oblong. The liquid barrier has a length and a width which may be selected in accordance with the type of barrier provided. In most situations it is desired that the width of the barrier is at least about 1 μm, such as at least about 5 μm, such as at least about 100 μm, such as up to about 2 mm. As mentioned it may be wider but in most situations this will merely be wasting space of the well.

The liquid barrier may be provided by any structure and/or surface properties which are capable of providing a resistance towards liquid to move from one of the first and the second essentially planar bottom surface sections to the other. In one embodiment the liquid barrier is provided by one or more of a low tension surface barrier, a ridge and an indentation.

The liquid barrier is arranged to provide an obstacle for liquid to pass from the first essentially planar bottom surface section to the second essentially planar bottom surface section.

In one embodiment the liquid barrier is or comprises a low tension surface barrier, which means that the surface tension of the well bottom in the liquid barrier is substantially lower than the surface tension of at least one, preferably both of respectively the first and the second essentially planar bottom surface sections. In one embodiment the surface of the well bottom in the liquid barrier has a surface tension which is at least about 5 mN/m, such as at least about 10 mN/m, such as between about 15 mN/m and about 60 mN/m lower than the surface tension of one or both of the first and the second essentially planar bottom surface sections. In one embodiment the surface of the well bottom in the liquid barrier has a surface tension which is up to about 75 mN/m, such as up to about 55 mN/m, such as between about 20 mN/m and about 65 mN/m.

Surface tension may e.g. be measured using contact angle. For a surface with a surface tension of less than about 73 mN/m the contact angle to water/sample is at least about 90 degrees measured in air at 20° C. All measurements are performed in air and at 20° C. and at atmospheric pressure unless anything else is mentioned.

The surface energy and the surface tension are two terms covering the same property of a surface and in general these terms are used interchangeably. The surface energy of a surface may be measured using a tensiometer, such as a SVT 20, Spinning drop video tensiometer marketed by DataPhysics Instruments GmbH. In this application the term ‘surface tension’ is the macroscopic surface energy, i.e. it is directly proportional to the hydrophilic character of a surface which may e.g. be measured by contact angle to a drop of water as it is well known to the skilled person. In comparing measurements, e.g. when measuring which of two surface parts has the highest surface energy, it is not necessary to know the exact surface energy and it may be sufficient to simply compare which of the two surfaces has the lower contact angle to water.

In one embodiment the liquid barrier is or comprises a ridge. The ridge has a height, which is the distance between the highest point of the ridge and the first essentially planar bottom surface section measured perpendicular to the first essentially planar bottom surface section. The height of the ridge should be sufficient to provide an obstacle for liquid to pass from the first essentially planar bottom surface section to the second essentially planar bottom surface section. In one embodiment the ridge has a height of at least about 5 μm, such as at least about 10 μm, such as at least about 50 μm, such as at least about 100 μm. In one embodiment the ridge has a height of from about 50 μm to about 1 mm, such as from about 100 μm to about 500 μm. The ridge should preferably not be higher than the well border edge, so that the cover can rest on the well border edge when applied onto the well plate. In one embodiment the ridge has a height which is essentially the same as the height of the well border edge.

In one embodiment the liquid barrier is or comprises an indentation, preferably in the form of a v shaped notch. In principle the indentation may have any other shape providing a sufficient obstacle for liquid to pass from the first essentially planar bottom surface section to the second essentially planar bottom surface section. It is desired that the indentation has relatively sharp edges to one or both of the first and the second essentially planar bottom surface sections. Such sharp edges e.g. about 145 degrees or less provide a capillary breach. This effect is well known to the skilled person and he will without unduly effort be able to provide an indentation which provides the desired barrier effect.

In one embodiment of the invention where the liquid barrier is oblong with a first and a second barrier end, said first and said second barrier end respectively have a shortest barrier-border distance to said well border or one or both of said first and said second barrier being coinciding with said well border edge, the length of the liquid barrier preferably being at least as long as any shortest barrier-border distance. Thereby the liquid barrier provides a very strong obstacle against passing of liquid over the liquid barrier.

The shortest barrier-border distance may in one embodiment be up to about 10 mm, such as up to about 5 mm, such as up to about 2 mm, such as up to about 1 mm, such as up to about 0.1 mm, preferably the shortest barrier-border distance being up to about 20% of the largest well bottom dimension measured from well border edge to well border edge.

In one embodiment of the invention the liquid barrier extends from well border edge to well border edge. This embodiment may be very simple to produce e.g. by using the two plate method described above wherein the first plate is provided by the liquid barrier prior to being fixed to the perforated plate.

In one embodiment of the invention the well comprises a second essentially planar bottom surface section in a second bottom plane. The second bottom plane may preferably be essentially parallel to said first bottom plane. In one embodiment the first essentially planar bottom surface section may be slightly inclined away from said second essentially planar bottom surface section to prevent liquid applied onto said first essentially planar bottom surface section from slipping down onto said second essentially planar bottom surface section.

The second essentially planar bottom surface section may accordingly in one embodiment be displaced with respect to said first essentially planar bottom surface section, such that said second bottom plane and said first top plane are essentially parallel and have a second bottom plane-cover distance which is larger than the first bottom plane-cover distance provided between said first bottom plane and said first top plane. The distance between two planes is measured perpendicularly to said planes.

In one embodiment of the invention the second bottom plane and the first top plane are essentially parallel and have a distance up to about 10 mm, such as from about 25 μm to about 1 mm, when the cover is applied onto said well plate. In principle the distance between the second bottom plane and the first top plane is not important for the function of the crystallization system, but off course for handling reasons the distance between the second bottom plane and the first top plane should neither be too large, nor too small.

In one embodiment of the invention the first planar bottom surface section provides a bottom surface of a first chamber and the second planar bottom surface section provides a bottom surface of a second chamber when the cover is applied onto the well e.g. when applied onto the well border edge. The first and the second chamber are in vapor connection with each other. This vapor connection may in principle be provided by any opening from the first to the second chamber. In one embodiment the first and the second chamber are interconnected with an interconnection opening which at its most narrow cross section is at least about 5 μm², such as at least about 10 μm², such as at least about 25 μm², such as at least about 50 μm².

In principle the interconnection opening may be as large as desired for example with a most narrow cross section of up to about 100 mm², such as with a most narrow cross section of from about 5 μm² to about 10 mm², such as from about 50 μm² to about 1 mm².

The planar bottom surface sections may in principle have any size, but for simplification and for having as many wells in each well plate as possible in order to make it simple to perform many tests simultaneously it is generally preferred to keep the sizes of the planar bottom surface sections relatively small. Often it is desired to use a relatively small droplet of target solution whereas the amount of precipitant is less critical and therefore it is often desired that the first planar bottom surface section which is for the target solution is smaller than the second planar bottom surface section which is for the precipitant. In one embodiment of the invention the first planar bottom surface section has a size of from about 10% to about 180% of said second planar bottom surface section.

In one embodiment of the invention the bottom surface is at least about 1 mm², such as at least about 9 mm², such as at least about 25 mm², such as at least about 50 mm², such as ^{from} about 25 mm² to about ⁴⁰⁰ mm².

In one embodiment of the invention the first essentially planar bottom surface section is at least about 1 mm², such as at least about 9 mm², such as at least about 25 mm², such as at least about 50 mm², such as from about 25 mm² to about 400 mm².

In one embodiment of the invention the second essentially planar bottom surface section is at least about 1 mm², such as at least about 9 mm², such as at least about 25 mm², such as at least about 50 mm², such as from about 25 mm² to about 400 mm².

In one embodiment of the invention the bottom surface has a smallest cross sectional dimension which is at least about 0.05 mm, such as at least about 1 mm, such as at least about 3 mm.

In one embodiment of the invention the first essentially planar bottom surface section has a smallest cross sectional dimension which is at least about 0.05 mm, such as at least about 1 mm, such as at least about 3 mm.

The well may in one embodiment have several second essentially planar bottom surface sections.

The well may have any shape. The shape of the well is defined by the well border edge of the well. Preferably the shape of the well should be such that the well border edge to well border edge is not too small to provide a capillary channel when the cover is applied. The well border edge to well border edge should in one embodiment not be less than about 100 μm, such as not less than about 1 mm, such as not less than 2 mm. The shape of the well may for example be round, circular, oval, square, rectangular, butterfly shaped or dumbbell shaped.

The first bottom plane-cover distance D₁ should preferably be sufficiently small to ensure that a droplet of target solution placed on the first essentially planar bottom surface section will be brought into contact with the first essentially planar top surface section when the cover is applied onto the well plate. In one example the first bottom plane-cover distance D₁ is up to about 800 μm, such as up to about 600 μm, such as up to about 500 μm.

In one embodiment the distance D₁ is from about 25 μm to about 900 μm, such as from about 50 μm to about 700 μm, such as from about 100 μm to about 500 μm. In a typical embodiment which will be useful for promoting crystallization of most macromolecules the distance D₁ is about 250 μm. In one embodiment it is preferred that the distance should be relatively small, i.e. smaller than the size of a droplet which is usually used for crystallization.

As indicated above it is often desired that the well plate comprises two or more wells in order to make it simple to perform several tests simultaneously. In one embodiment of the invention the well plate comprises a plurality of identical or different wells, such as at least 4 wells, such as at least 50 wells, such as at least 68 wells. The wells may preferably be arranged in a regular pattern so that it is possible and simple to place the droplets of target solution(s) and precipitant(s) using a robot.

In one embodiment of the invention at least a section of said cover is transparent to provide at least a transparent window into the well, preferably said cover or a part thereof being of a transparent material. The transparent material should be transparent to electromagnetic waves of at least one wavelength. In principle this one or more wavelengths to which the transparent material is transparent may be any wavelength. In a preferred embodiment the transparent material is transparent to at least one wavelength selected from Infrared light (about 700 nm to about 1000 μm), visibly light (about 400 nm to about 700 nm), UV light (about 400 nm to about 10 nm) about and X-ray light (about 10 nm to about 0.01 nm). In a preferred embodiment the transparent material is transparent to a range of wavelengths. In one embodiment of the invention the transparent material is transparent to a range of wavelengths in the short range area such as a range of wavelengths selected from the wavelength from about 0.01 nm to about 700 nm. By using short wave light in the analysis, the analysis of the crystallized structure of the target molecule can be very detailed and the degrading of the crystal due to heat generation is very small if any at all.

In one embodiment of the invention the cover is arranged to provide a tightening between said cover and said well border edge when the cover is applied onto the well plate. The tightening need not to be absolutely tight but it should be sufficiently tight to perform the crystallization without risk of undesired contamination.

The cover may in one embodiment be of a relatively stiff material, i.e. a material that does not collapse due to gravity forces. In one embodiment the cover is a shaped cover comprising an outer edge for positioning onto said well plate.

In one embodiment the cover is a plate, e.g. a simple planar plate such as a slide.

In one embodiment of the invention the cover is a film, e.g. a flexible film which can be stretched over well plate.

The cover may in principle have any thickness. However in situations where at least a part of the cover is transparent such that the crystallized structure of the target molecule may be observed and optionally analyzed over said cover without removing the cover, it will often be desired that the cover is not too thick in order to provide the cover as transparent as possible. In one embodiment of the invention the cover has a thickness of from about 1 μm to about 1 mm, such as from about 25 μm to about 700 μm, such as from about 50 μm to about 500 μm, such as from about 100 μm to about 200 μm.

Examples of useful materials for the well and/or the cover include glass and polymer, preferably a polymer selected from cyclic oleofin copolymers (COC), acrylonitrile-butadiene-styrene copolymer, polycarbonate, polydimethylsiloxane (PDMS), polyethylene (PE), polymethylmethacrylate (PMMA), polymethylpentene, polypropylene, polystyrene, polysulfone, polytetrafluoroethylene (PTFE), polyurethane (PU), polyvinylchloride (PVC), polyvinylidene chloride (PVDC), polyvinylidine fluoride, styrene-acryl copolymers polyisoprene, polybutadiene, polychloroprene, polyisobutylene, poly(styrene-butadiene-styrene), silicones, epoxy resins, Poly ether block amide, polyester, acrylonitrile butadiene styrene (ABS), acrylic, celluloid, cellulose acetate, ethylene-vinyl acetate (EVA), ethylene vinyl alcohol (EVAL), fluoroplastics, polyacetal (POM), polyacrylates (acrylic), polyacrylonitrile (PAN) polyamide (PA), polyamide-imide (PAI), polyaryletherketone (PAEK), polybutadiene (PBD), polybutylene (PB), polybutylene terephthalate (PBT), polyethylene terephthalate (PET), polycyclohexylene dimethylene terephthalate (PCT), polyketone (PK), polyester/polythene/polyethene, polyetheretherketone (PEEK), polyetherimide (PEI), polyethersulfone (PES), polyethylenechlorinates (PEC), polyimide (PI), polylactic acid (PLA), polymethylpentene (PMP), polyphenylene oxide (PPO), polyphenylene sulfide (PPS), polyphthalamide (PPA), and mixtures thereof.

The one or more surfaces of respectively the well(s) and the cover may be subjected to a treatment which alters the surface tension e.g. a corona treatment, a plasma treatment a VPD (Vapor deposition treatment) and/or a deposition treatment e.g. for deposition of a coating comprising chemically active components and/or a colored element which preferably may be transparent for UV and/or X-ray light. Thereby the crystallized molecules may be simpler to observe visually, in particular if at least one window into the well is transparent to visual light, and simultaneously a detailed analysis of the molecule may be obtained by UV and/or X-ray light.

In a preferred embodiment at least a part of at least one of said well and said cover is made from an X-ray transparent material, preferably a polyimide, e.g. a phenylene-pyromellitimide such as poly(4,4′-oxodiphenylene-pyromellitimide e.g. Kapton®.

It has been found that when using an X-ray transparent material for at least a part of at least one of said well and said cover it is extremely simple to observe and analyze the crystallized structure of the target molecule without removing the cover. By using X-ray for analyzing the crystallized structure of the target molecule a very detailed analysis can be obtained without substantial damaging of the crystallized molecule.

In one embodiment of the invention at least the cover is made from an X-ray transparent material, preferably a polyimide, e.g. a phenylene-pyromellitimide such as poly(4,4′-oxodiphenylene-pyromellitimide e.g. Kapton®. The cover may in this embodiment for example be of a film of the X-ray transparent material.

In one embodiment of the invention the crystallization system comprises a well plate and a cover for the well plate, the well plate may e.g. be as described above. In this embodiment at least one of said well plate and said cover comprises at least a transparent window as described above, and the well plate comprises at least one well comprising a bottom surface comprising a first essentially planar bottom surface section in a first bottom plane and a well border wall provided by a well border edge surrounding the planar bottom surface section and lying in an edge plane, such that the first bottom plane and said edge plane are essentially parallel and having a ‘first bottom plane-edge plane distance’ D₂ of from about 10 μm to about 1000 μm, such as up to about 800 μm, such as up to about 600 μm, such as up to about 500 μm.

In one embodiment the distance D₂ is from about 25 μm to about 900 μm, such as from about 50 μm to about 700 my, such as from about 100 μm to about 500 μm. In a typical embodiment which will be useful for promoting crystallization of most macromolecules the distance D₂ is about 250 μm. In one embodiment it is preferred that the distance should be relatively small, i.e. smaller than the size of a droplet which is usually used for crystallization.

In one embodiment D₁ is substantially identical to D₂.

The well plate and/or the cover may further be equipped with a temperature control unit such as a peltier element.

In one embodiment the crystallization system further comprises a holder for holding the well plate e.g. during loading and/or during analysis.

In one embodiment the crystallization system comprises a holder for holding and for mounting the well plate with the cover on or in an imaging system and/or in front of an electromagnetic wave source, such as an X-ray source. Such a holder is in particular useful when the well plate is relatively thin and/or small.

As explained below it may sometimes be desirable to cut-out part of the well plate with the cover on for further analysis using X-rays or other wavelengths. In one embodiment the crystallization system comprises a holder for holding and for mounting cut-out sections of the well plate with cover in an imaging system and/or in front of an electromagnetic wave source, such as an X-ray source.

A cutting tool may be specially designed for cutting out part of the well plate with cover, e.g. designed to cut-out a section of the well plate with cover having a few wells or only one single well.

The invention also relates to a kit comprising a crystallization system and a test liquid. The crystallization system comprises a well plate and a cover for said well plate. At least one of said well plate and said cover comprises at least a transparent window as described above. The well plate comprises at least one well comprising a bottom surface comprising a first essentially planar bottom surface section in a first bottom plane and a well border wall provided by a well border edge surrounding said planar bottom surface section. The cover comprises a first essentially planar top surface section in a first top plane adapted to face said first essentially planar bottom surface section, such that said first bottom plane and said first top plane are essentially parallel and have a ‘first bottom plane-first top plane distance’ D₃ when the cover is applied onto said well border edge. The first bottom plane-first top plane distance D₃ is smaller than the height of the test liquid when it is applied as a droplet onto said first essentially planar bottom surface section.

In one embodiment of the invention the crystallization system is as described above and D₃ is identical to D₁.

In one embodiment of the invention the crystallization system of the kit is as described above with the exception that the first bottom plane-first top plane distance is larger than D₁

The test liquid applied to the first essentially planar bottom surface section may preferably be a target molecule solution. The target molecule may in principle be any kind of molecules which can crystallize from a solution, which solution will often have a relatively high concentration of the target molecule.

In particular it is often desired that the target molecule is a macromolecule, preferably having a size of at least about 10 Angstroms, such as at least about 100 Angstroms. The macromolecule may for example have a molar mass of from about 1,000 to about 1,000,000. For proteins the size will most often be from about 10,000 to about 200,000 Dalton (corresponding to molar mass)

The target molecule may be organic or inorganic. Most often the target molecule will be a biomolecule i.e. molecules which originate from a biological specimen or artificial analogues thereto. Preferably the target molecule solution is a solution of at least one kind of target molecules selected from the group consisting of proteins, nucleic acids, nucleic acids analogues, carbohydrates, lipids more preferably selected from the group of proteins of 500 Dalton or more, single and double stranded DNA, RNA, PNA and LNA, and drug candidates. The term protein includes peptides as well as larger proteins.

In a preferred embodiment the target molecule is a protein selected from the group of protein of 500 Dalton or more.

In one embodiment the target molecule solution comprises two or more molecules which may react to form the desired crystallized molecule.

As indicated above the solution may comprise other elements e.g. for stabilizing the solution, e.g. polymer such as polyethylene glycol, and surfactant including detergents.

The concentration of the various elements may vary largely.

Examples of detergents and concentration are found in table 1. Usually a target molecule solution will comprise only one type of detergent; however combinations of detergents may also be applied.

MW: molecular weight.
CMC: critical micelle concentration.
Actual: typical concentration used.

TABLE 1

| Detergent | MW | CMC (mM) | [Actual] (mM) | |
|---|---|---|---|---|
| C12E9 | 583.10 | 0.1 | 0.8 | |
| C12E8 | 539.10 | 0.1 | 1.1 | |
| n-Dodecyl-β-D-maltoside | 510.60 | 0.2 | 1.7 | |
| Sucrose monolaurate | 524.60 | 0.2 | 2.0 | |
| CYMAL ®-6 | 508.50 | 0.6 | 5.6 | |
| TRITON ® X-100 | 631.00 | 0.9 | 9.0 | |
| CTAB | 364.50 | 1.0 | 10.0 | |
| Deoxy BigChap | 862.10 | 1.4 | 14.0 | |
| n-Decyl-β-D-maltoside | 482.60 | 1.8 | 18.0 | |
| LDAO | 229.40 | 2.0 | 20.0 | |
| CYMAL ®-5 | 494.50 | 2.4 | 24.0 | |
| ZWITTERGENT ® 3-12 | 335.60 | 4.0 | 40.0 | |
| Nonyl-β-D-glucoside | 306.40 | 6.5 | 65.0 | |
| 1-s-Octyl-β-D-thioglucoside | 308.40 | 9.0 | 90.0 | |
| DDAO | 201.40 | 10.4 | 104.0 | |
| HECAMEG | 335.40 | 19.5 | 195.0 | |
| n-Octanoylsucrose | 468.50 | 24.4 | 244.0 | |
| Heptyl-β-D-thioglucoside | 274.30 | 30.0 | 300.0 | |
| n-Octyl-β-D-glucoside | 292.40 | 24.5 | 245.0 | |
| CYMAL ®-3 | 466.50 | 34.5 | 345.0 | |
| C-HEGA-10 | 377.50 | 35.0 | 350.0 | |
| ZWITTERGENT ® 3-10 | 307.60 | 40.0 | 400.0 | |
| MEGA-8 | 321.40 | 79.0 | 790.0 | |
| n-Hexyl-β-D-glucoside | 264.30 | 250.0 | 2500.0 | |
| Pluronic ® F-68 | ~8350 | None | 10% | w/v |
| Anapoe ® 35 | None | None | 10% | v/v |
| n-Dodecyl-β-D-maltotrioside | 672.78 | 0.2 | 2 | mM |
| Anapoe ® 58 | None | None | 10% | v/v |
| Anapoe ® X-114 | None | None | 10% | v/v |
| Anapoe ® X-305 | None | None | 10% | v/v |
| Anapoe ® X-405 | None | None | 10% | v/v |
| Anapoe ® 20 | 1227.54 | 0.059 | 10% | v/v |
| Anapoe ® 80 | 1309.68 | 0.012 | 10% | v/v |
| Anapoe ® C10E6 | 427.10 | 0.9 | 10% | v/v |
| Anapoe ® C10E9 | None | None | 10% | v/v |
| Anapoe ® C12E10 | None | None | 10% | v/v |
| Anapoe ® C13E8 | None | None | 10% | v/v |
| IPTG | 238.30 | None | 10% | w/v |
| n-Dodecyl-N,N- | 271.40 | 1.5 | 15.0 | mM |
| dimethylglycine | | | | |
| HEGA-10 | 379.50 | 7.0 | 70.0 | mM |
| C8E5 | 350.50 | 7.1 | 71.0 | mM |
| CHAPS | 614.90 | 8.0 | 80.0 | mM |
| CHAPSO | 630.90 | 8.0 | 80.0 | mM |
| C-HEGA-11 | 391.50 | 11.5 | 115 | mM |
| HEGA-9 | 365.50 | 39.0 | 390 | mM |
| C-HEGA-9 | 363.50 | 108.0 | 1.08M | |
| HEGA-8 | 351.50 | 109.0 | 1.09M | |
| CYPFOS-3 | 293.30 | 180.0 | 1.80M | |
| BAM | 384.45 | None | 10% | w/v |
| n-Hexadecyl-β-D-maltoside | 566.6 | 0.0006 | 0.006 | mM |
| n-Tetradecyl-β-D-maltoside | 538.6 | 0.01 | 0.1 | mM |
| n-Tridecyl-β-D-maltoside | 524.6 | 0.033 | 0.33 | mM |
| Thesit ® | 582.9 | 0.09 | 0.9 | mM |
| Zwittergent ® 3-14 | 363.6 | 0.4 | 4.0 | mM |
| n-Undecyl-β-D-maltoside | 496.6 | 0.59 | 5.9 | mM |
| n-Decyl-β-D-thiomaltoside | 498.6 | 0.9 | 9.0 | mM |
| FOS-Choline ®-12 | 315.5 | 1.5 | 15.0 | mM |
| n-Decanoylsucrose | 496.6 | 2.5 | 25 | mM |
| 1-s-Nonyl-β-D-thioglucoside | 322.4 | 2.9 | 29.0 | mM |
| n-Nonyl-β-D-maltoside | 484.6 | 3.2 | 32.0 | mM |
| DDMAB | 299.5 | 4.3 | 43.0 | mM |
| n-Nonyl-β-D-maltoside | 468.4 | 6 | 60.0 | mM |
| Cymal ®-4 | 480.5 | 7.6 | 76.0 | mM |
| n-Octyl-β-D-thiomaltoside | 470.6 | 9 | 90.0 | mM |
| FOS-Choline ®-10 | 323.4 | 13 | 130 | mM |
| FOS-Choline ®-9 | 309.4 | 19 | 190 | mM |
| MEGA-9 | 335.5 | 25 | 250 | mM |
| 1-s-Heptyl-β-D-thioglucoside | 294.4 | 29 | 290 | mM |

| | | | |
|---|---|---|---|
| FOS-Choline ®-8 | 295.4 | 102 | 1.02M |
| Cymal ®-2 | 452.5 | 120 | 1.20M |
| Zwittergent ®-3-08 | 279.6 | 330 | 3.30M |
| Cymal ®-1 | 438.5 | 340 | 3.4M |

In one embodiment a gel-forming material may be added to the target molecule solution to stabilize the crystals when formed. Examples of useful gel-forming materials are agarose and acrylamide.

The test liquid applied to the first essentially planar bottom surface section may in principle have any desired volume. As mentioned above it is often desired to use a relatively low volume. There may be several reasons for this, for example the molecule may be expensive, the molecule may form a more optimal crystal when the volume is low and the molecule in crystallized form may be easier to analyze. In one embodiment the volume of the test liquid applied to the first essentially planar bottom surface may be from about 1 nL to about 100 μL.

In one embodiment the test liquid is a target molecule solution comprising at least a dissolved target molecule, a solvent and optionally a precipitant.

In one embodiment the test liquid has a surface tension of at least 60 mN/m, the test liquid may preferably be an aqueous solution.

In one embodiment the test liquid is an aqueous solution further comprising a surfactant, such as a detergent.

Besides the above description of test liquids, the test liquid may be as described in prior art e.g. as in the prior art referred to above.

In one embodiment the kit further comprises a separate precipitant.

The term “precipitant” is used to denote any components, compositions and substances that cause or help a target molecule to precipitate. The precipitant will usually be in a liquid form. The term “precipitant” includes any solutions thereof.

Useful precipitants and combinations of precipitants are well known from the art. As specified above the precipitant may be applied in dry form or in a solution.

Examples of precipitant solution can be found in Shotgun crystallization strategy for structural genomics: an optimized two-tiered crystallization screen against the Thermotoga maritima proteome’ by Page R, Grzechnik S K, Canaves J M, Spraggon G, Kreusch A, Kuhn P, Stevens R C, Lesley S A. ACTA CRYSTALLOGRAPHICA SECTION D-BIOLOGICAL CRYSTALLOGRAPHY 59: 1028-1037 Part 6, JUN 2003

The skilled person will know how to find and to select the precipitant for use in combination with selected target molecule solution.

The invention also relates to a method of promoting crystallization of a target molecule. The method of the invention comprises
providing a test liquid sample comprising a solution of a target molecule;
providing a crystallization system comprising a well plate and a cover for said well plate, at least one of said well plate and said cover comprises at least a transparent window, said well plate comprises at least one well comprising a bottom surface comprising a first essentially planar bottom surface section and said cover comprises a first essentially planar top surface section,
applying said test liquid sample onto said first essentially planar bottom surface section;
applying said cover onto said well plate, such that said first essentially planar top surface section is in physical contact with said test liquid sample; and
allowing said target molecule to crystallize.

The crystallization system and/or kit may be as described above.

In one embodiment of the method of the invention the crystallization system is as the double chamber crystallization system described below.

In one embodiment of the method the test liquid is as described above.

In one embodiment the method comprises applying the test liquid sample in the form of a droplet onto said first essentially planar bottom surface section. The droplet of the test liquid may in principle have any volume but as mentioned above a volume of about 100 μL or less is desired. In one embodiment the test liquid sample is applied in the form of a droplet with a volume of from about 1 nL to about 100 μL, such as from about 20 nL to about 10 μL, such as from 50 nL to about 1 μL, such as from 100 nL to about 500 nL.

In one embodiment the method of the invention comprises applying a precipitant in contact with said test liquid sample. The precipitant may for example be placed immediately adjacent to and in contact with the test liquid sample or it may be applied onto the test liquid sample, preferably in the form of a droplet.

In one embodiment where the method comprises applying a precipitant in contact with said test liquid sample the precipitant and the test liquid sample are applied simultaneously e.g. by being pre-mixed or being applied from an applicator with laminar flow, the two liquids are flowing in separate laminae/lamina of the laminar flow.

In one embodiment where the method comprises applying a precipitant in contact with said test liquid sample the precipitant and the test liquid sample are applied one after the other i.e. first applying the and onto the precipitant applying the test liquid sample or first applying the test liquid sample, and onto the test liquid sample applying the precipitant.

In one embodiment the method of the invention comprises applying a precipitant side by side with said test liquid sample without being in physical contact with each other, both of the precipitant and the test liquid sample are in one embodiment applied to first essentially planar bottom surface section. The order of applying the precipitant and the test liquid sample is not important. and they may for example be applied simultaneously or within a short interval, such than less than about 30 minutes, preferably less than about 5 minutes. The crystallization will take place via a vapor diffusion method.

In one embodiment wherein the crystallization system comprises a second essentially planar bottom surface section, the method comprises applying a precipitant onto said second essentially planar bottom surface section. In this embodiment the test liquid sample and the precipitant will not be in physical contact, but the crystallization will take place via a vapor diffusion method.

The precipitant may be as described above.

The precipitant may be applied in the form of a droplet. The size of the precipitant droplet is not so important as long as it has a sufficient surface area. In one embodiment the precipitant droplet is larger than the test liquid droplet. In one embodiment the precipitant droplet is about 1 mL or less, such as from about 1 nL to about 100 μL.

The test solution droplet and/or the precipitant droplet may e.g. be applied using a tool, such as a pipette. In one embodiment the test solution droplet and/or the precipitant droplet may e.g. be applied using an automated or semi-automated fluid manipulation system such as a robot.

In one embodiment the precipitant(s) are pre-filled into the well of the crystallization system, preferably by being applied onto the second planar bottom surface section. The precipitant may in this embodiment preferably be in a dried state. The dry precipitant may be re-dissolved prior to or after application of the test liquid sample e.g. by applying a solvent for the precipitant onto the dry precipitant on the second planar bottom surface section.

In order to prevent liquid from evaporating from the crystallization system the crystallization system may be sealed e.g. by applying a sealing element/material between the well edge(s) and the cover.

Any sealing element/material may be used. In one embodiment the crystallization system may be sealed e.g. by

a) adding a wax, such as a paraffin wax or a polyethylene wax to seal between the well edge(s) and the cover; or
b) fixing (e.g. by gluing, welding or clamping) the well edge(s) to the cover.

The method may preferably further comprise incubating the crystallization system and allowing crystals to be formed and/or to grow. The incubating time depends on the type of target molecule solution. In general the most typical incubating times will be between 2 and 580 hours, such as between 24 and 240 hours. The incubation typically takes place in temperature controlled boxes, e.g. with a temperature of 25 degrees, 16 degrees, or 4 degrees. The temperature may influence the crystallization and for some tests, incubation at varying temperatures may be performed.

After incubation the crystallization system is inspected e.g. visually or by a robot, to identify any crystal formation. The formed crystals may be examined in the liquid channel e.g. through a transparent wall section or they may be harvested for further examination.

In one embodiment the method of the invention further comprises
observing if said target molecule crystallizes, and if so
analyzing said crystallized target molecule.

The analyzing preferably is performed optically, more preferably by X-ray. The optical analysis may preferably be performed by absorption, scattering and/or diffraction. When using X-ray diffraction analysis is often preferred.

In one embodiment the method of the invention comprises placing the well plate in a holder and holding the well plate e.g. during loading and/or during analysis.

In one embodiment the method of the invention comprises placing the well plate with the cover on in a holder for holding and for mounting the well plate with the cover on or in an imaging system and/or in front of an electromagnetic wave source, such as an X-ray source. Such a holder is in particular useful when the well plate is relatively thin and/or small.

In one embodiment the method of the invention comprises cutting the well plate with the cover into one or more cut-out sections, each cut-out section comprising a few of the wells of the well plate with cover, such a one row of wells, such 5 wells or less, such as one single well. These cut-out parts of the well plate with the cover on may be subjected to further analysis using X-rays or other wavelengths. In one embodiment the method comprises placing a cut-out part of the well plate with cover in a holder and mounting it in an imaging system and/or in front of an electromagnetic wave source, such as an X-ray.

In one embodiment the method of the invention further comprises cooling or freezing, preferably flash cooling/freezing the cut-out part or the entire well plate in a cooling medium such as liquid nitrogen. In this embodiment the method of the invention may further comprise adding a chemical that protects the crystal during the cooling process prior to cooling, e.g. prior to crystallization.

In another aspect of the invention it relates to a double chamber crystallization system comprising a well plate and a removable cover for the well plate. At least one of said well plate and the cover comprises at least a transparent window which may be as described for the crystallization system above. The well plate comprises at least one well comprising a first essentially planar bottom surface section and a second essentially planar bottom surface section, and a well border wall provided by a well border edge surrounding said first and said second essentially planar bottom surface sections. The first essentially planar bottom surface section and the second essentially planar bottom surface section are arranged in a common bottom plane, wherein the first planar bottom surface section provides a bottom surface of a first chamber and the second planar bottom surface section provides a bottom surface of a second chamber when the cover is applied onto the well plate, and the first and the second chamber are in vapor communication with each other.

In one embodiment of the double chamber crystallization system the cover is arranged to be placed onto the well plate to provide a tightening between said cover and said well border edge. The cover may for example be as described for the crystallization system above.

In one embodiment of the double chamber crystallization system the first and the second chambers are interconnected with an interconnection opening which at its most narrow cross section is at least about 5 μm², such as at least about 10 μm², such as at least about 25 μm², such as at least about 50 μm².

In principle the interconnection opening may be as large as desired for example with a most narrow cross section of up to about 100 mm², such as with a most narrow cross section of from about 5 μm² to about 10 mm², such as from about 50 μm² to about 1 mm².

In one embodiment of the double chamber crystallization system at least a section of at least one of said bottom wall and said cover is transparent to provide at least a transparent window into at least said first chamber, preferably at least one of said cover and said bottom wall is of a transparent material, said material preferably being transparent to Infrared light (about 700 nm to about 1000 μm), visibly light (about 400 nm to about 700 nm), UV light (about 400 nm to about 10 nm) about and/or X-ray light (about 10 nm to about 0.01 nm).

In one embodiment at least one of the well plate and the cover is totally or partly made from one or more of the materials described above for the crystallization system and one or more surfaces thereof may be treated as also described above.

In one embodiment of the double chamber crystallization system the cover comprises a top surface section adapted to face said first and said second essentially planar bottom surface sections, the minimum distance D₄ between respectively the first and the second essentially planar bottom surface sections and the top surface section is at least about 1 mm, such as at least about 2 mm, such as from about 3 mm to about 20 mm, when the cover is applied onto said well plate.

In one embodiment of the double chamber crystallization system the first and the second essentially planar bottom surface sections are totally or partly separated by a liquid barrier, said liquid barrier preferably being oblong and having a length and a width, the width preferably being at least about 1 μm, such as at least about 5 μm, such as at least about 100 μm, such as up to about 2 mm.

In one embodiment of the double chamber crystallization system the liquid barrier is provided by one or more of a low tension surface barrier, a ridge and an indentation.

The liquid barrier may be as the liquid barrier described for the crystallization system above.

In one embodiment of the double chamber crystallization system the liquid barrier is oblong with a first and a second barrier end, the first and said second barrier end respectively have a shortest barrier-border distance to or are coinciding with said well border edge, the length of the liquid barrier preferably being at least as long as any shortest barrier-border distance.

In one embodiment the shortest barrier-border distance is up to about 10 mm, such as up to about 5 mm, such as up to about 2 mm, such as up to about 1 mm, such as up to about 0.1 mm, preferably the shortest barrier-border distance is up to about 20% of the largest well bottom dimension measured from well border edge to well border edge.

In one embodiment of the double chamber crystallization system the liquid barrier extends from well border edge to well border edge.

The sizes of respectively the first planar bottom surface section and the second planar bottom surface section may preferably be as described for the crystallization system above.

The first and second planar bottom surface sections may independently of each other have any size. For practical reasons the first and second planar bottom surface sections independently of each other have a size of from about 9 mm² to about 400 mm².

In one embodiment the first and second planar bottom surface sections independently of each other have a smallest cross sectional dimension which is at least about 0.05 mm, such as at least about 1 mm, such as at least about 3 mm. Thereby a droplet may be applied onto respectively the first and second planar bottom surface sections without being subjected to capillary forces due to the surrounding well border wall.

The well may have any shape such as the shapes described above for the wells(s) of the crystallization system e.g. a shape defined by the well border edge of the well, which shape is round, circular, oval, square, rectangular, butterfly shaped or dumbbell shaped.

The well plate may comprise a plurality of identical or different wells, which wells e.g. may be arranged in a regular pattern.

The cover may be as described above for the crystallization system. In one embodiment the cover is a shaped cover comprising an outer edge for positioning onto said well plate. In one embodiment the cover is a plate. In one embodiment the cover is a film. In one embodiment the cover has a thickness of from about 1 μm to about 1 mm, such as from about 25 μm to about 700 μm, such as from about 50 μm to about 500 μm, such as from about 100 μm to about 200 μm.

The cover and the well of the double chamber crystallization system may in one embodiment, independently of each other be provided by the material disclosed above

In one embodiment the double chamber crystallization system further comprises a holder for holding the well plate e.g. during loading and/or during analysis.

In one embodiment the double chamber crystallization system comprises a holder for holding and for mounting the well plate with the cover on or in an imaging system and/or in front of an electromagnetic wave source, such as an X-ray source. Such a holder is in particular useful when the well plate is relatively thin and/or small.

As explained below it may sometimes be desirable to cut-out part of the well plate with the cover on for further analysis using X-rays or other wavelengths. In one embodiment the double chamber crystallization system comprises a holder for holding and for mounting cut-out sections of the well plate with cover in an imaging system and/or in front of an electromagnetic wave source, such as an X-ray source.

### BRIEF DESCRIPTION OF DRAWINGS

Embodiments of the invention will be described more fully below and with reference to the drawings in which:

FIG. 1a shows a perspective view of a first crystallization system of the invention comprising a well plate and a cover.

FIG. 1b shows the crystallization system of FIG. 1a with the cover applied onto the well plate.

FIG. 2a shows a perspective view of a second crystallization system of the invention comprising a well plate and a cover.

FIG. 2b shows the crystallization system of FIG. 2a with the cover applied onto the well plate.

FIG. 3a shows a perspective and enlarged view of a third crystallization system of the invention comprising a well plate and a cover.

FIG. 3b shows the crystallization system of FIG. 3a with the cover applied onto the well plate.

FIGS. 4a-d show a well of a crystallization system of the invention in 4 different stages of use.

FIGS. 5a-d show a well with a double chamber structure of a crystallization system of the invention in 4 different stages of use.

FIG. 6a shows a perspective view of a fourth crystallization system of the invention comprising parts of a well plate and a cover.

FIG. 6b shows the crystallization system of FIG. 6a with the cover applied onto the well plate.

FIG. 7a shows a perspective view of a fifth crystallization system of the invention comprising a well plate and a cover.

FIG. 7b shows the crystallization system of FIG. 7a with the cover applied onto the well plate.

FIGS. 8a-8i are schematic illustrations of 9 different well shapes.

FIG. 9 is a schematic view of a crystallization system of the invention comprising a crystallized molecule which is subjected to an optical analysis.

The figures are schematic and simplified for clarity and just show details which are essential to the understanding of the invention, while other details are left out. Throughout, the same reference numerals are used for identical or corresponding parts.

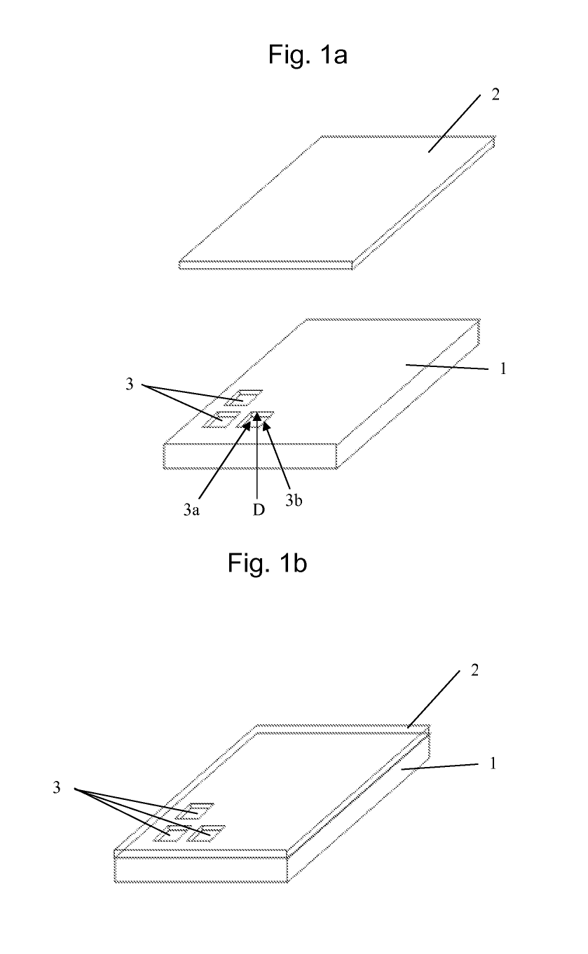

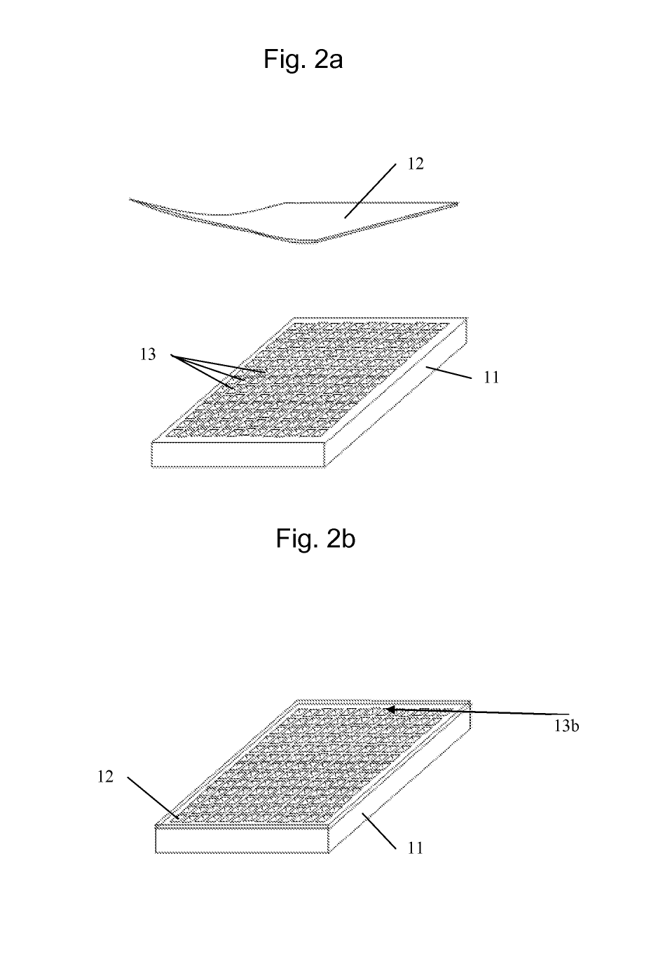

FIGS. 1a and 1b illustrate a first crystallization system of the invention comprising a well plate 1 and a cover 2. In FIG. 1a the well plate 1 and the cover 2 are separated from each other, and in FIG. 1b the cover 2 is applied onto the well plate 1. The well plate 1 comprises a plurality of wells 3, where only 3 are visible. The wells 3 are square but they could have other shapes as described above. Each well has a bottom surface which constitutes the first essentially planar bottom surface section and is indicated with the arrow 3a, and a well border edge 3b. The well border edge has a height D from the first essentially planar bottom surface section 3a, which is preferably as D₁, D₂ and/or D₃ described above. The cover 2 is a plate of a relatively stiff material. As it can be seen in FIG. 1b at least a part of the cover 2 is transparent to visible light. As mentioned above the cover 2 may preferably be made from an X-ray transparent material. In FIG. 1b where the cover 2 is applied onto the well plate 1a not shown sealing material e.g. a wax may be applied between the well plate 1 and the cover 2 to provide a sealing. Alternatively or additionally a not shown clamping device may be applied to hold the well plate 1 and the cover 2 together.

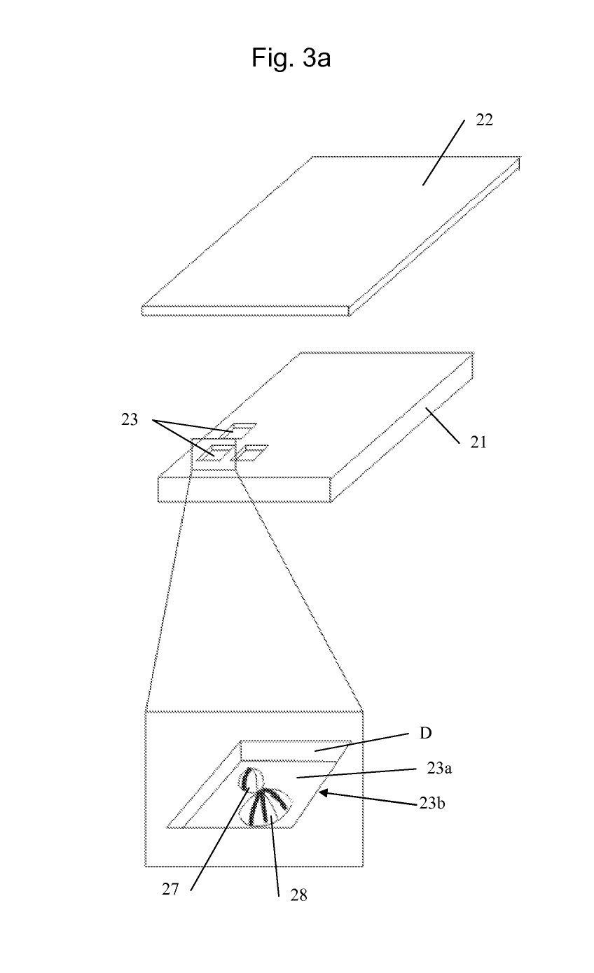

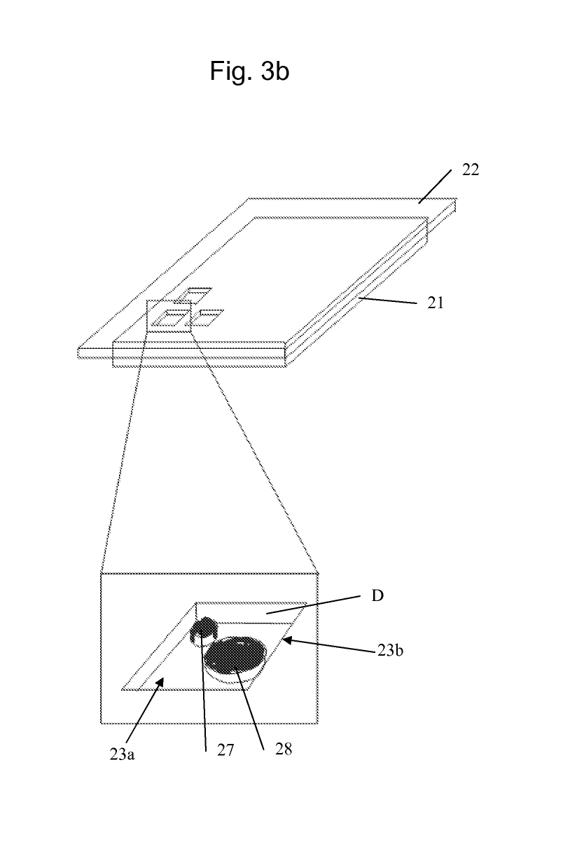

FIGS. 2a and 2b illustrate a second crystallization system of the invention which is similar but not identical to the first crystallization device shown in FIGS. 1a and 1b. The second crystallization system shown in FIGS. 2a and 2b comprises a well plate 11 and a cover 12. In FIG. 2a the well plate 11 and the cover 12 are separated from each other, and in FIG. 2b the cover 12 is applied onto the well plate 11. The well plate 11 comprises a plurality of wells 13. Each well has a not shown first essentially planar bottom surface section, and a well border edge 13b. The well border edge 13b has a height D from the first essentially planar bottom surface section, which is preferably as D₁, D₂ and/or D₃ described above. The cover 12 is a transparent—preferably X-ray transparent—film of a flexible material. The well plate 11 and the cover 12 may be sealed to each other as described above.

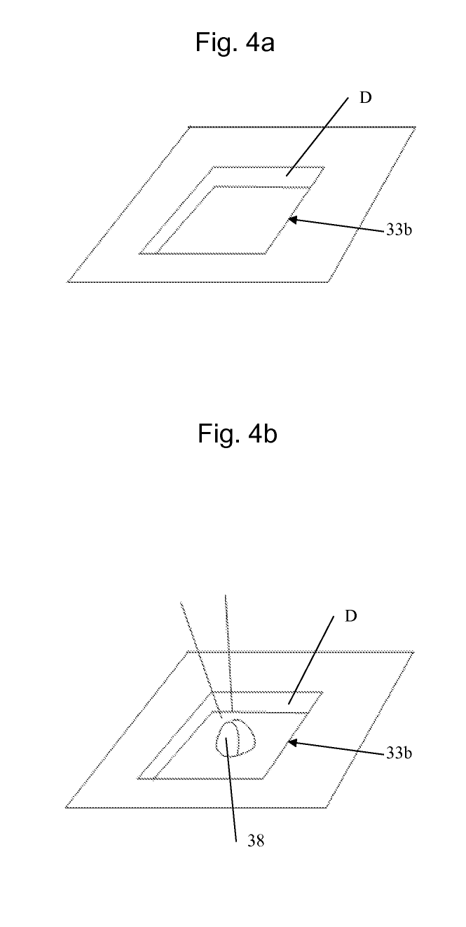

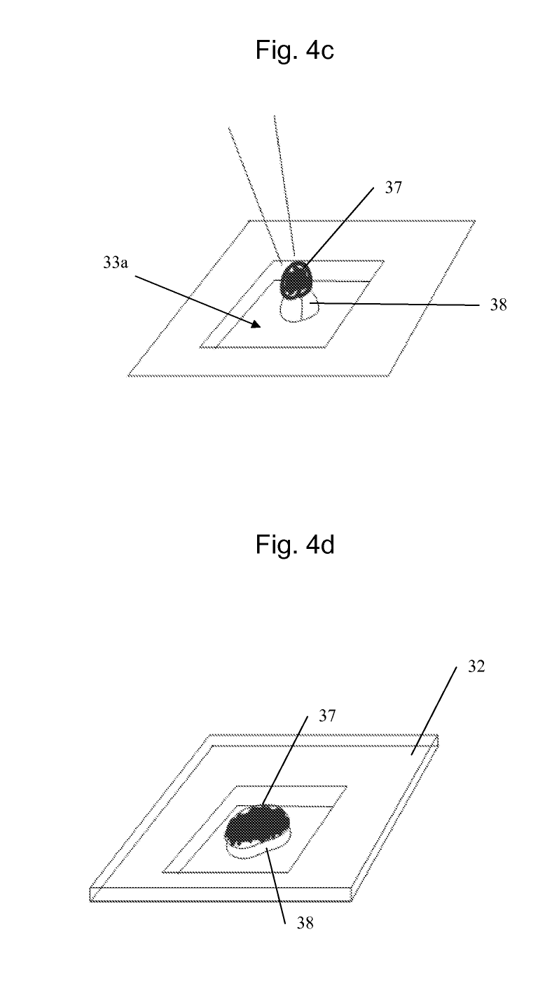

FIGS. 3a and 3b illustrate a third crystallization system of the invention which is similar and may be but not identical with the first crystallization device shown in FIGS. 1a and 1b. The third crystallization system shown in FIGS. 3a and 3b comprises a well plate 21 and a cover 22. In FIG. 3a the well plate 21 and the cover 22 are separated from each other, and in FIG. 3b the cover 22 is applied onto the well plate 21. The well plate 21 comprises a plurality of wells 23. Each well has a first essentially planar bottom surface section 23a, and a well border edge 23b. The well border edge 23b has a height D from the first essentially planar bottom surface section, which is preferably as D₁, D₂ and/or D₃ described above. The cover 22 is a transparent—preferably X-ray transparent—plate which has a slightly larger circumference than the well plate 21 and therefore extends beyond the border of the well plate 21 for providing simpler handling. The well plate 21 and the cover 22 may be sealed to each other as described above.

A section of respective FIGS. 3a and 3b showing one of the wells 23 is enlarged in the enlarged sections 25 and 26. In these enlarged sections 25 and 26 a use of the crystallization system is illustrated. In the enlarged section 25 of FIG. 3a a droplet of a test liquid 27 and a droplet of a precipitant 28 are applied onto the first essentially planar bottom surface section in a distance from each other. When the cover 23 is applied as shown in the enlarged section 26 of FIG. 3b the top surface of the cover 22 comes into physical contact with the droplet of test liquid 27 and the droplet of a precipitant 28 and presses these droplets slightly down to provide a relatively large contact area between the respective droplets and the top surface. Thereby analysis of a crystallized structure of the test liquid 27 is simple to perform optically without the need of removing the cover.

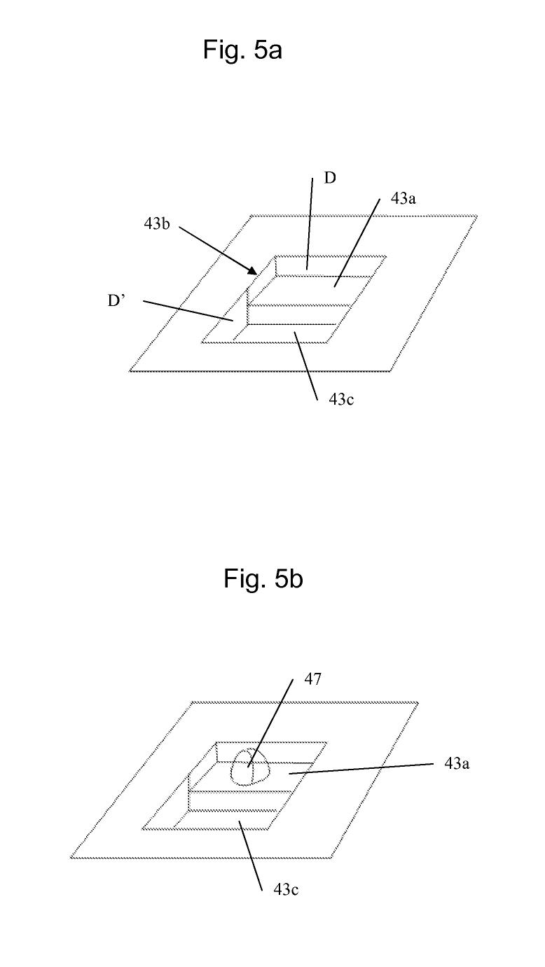

FIGS. 4a-d shows a well of a crystallization system which could be as any of the above described crystallization system, in 4 different stages of use. The well has a first essentially planar bottom surface section 33a, and a well border edge 33b. The well border edge 33b has a height D from the first essentially planar bottom surface section, which is preferably as D₁, D₂ and/or D₃ described above. In FIG. 4a the well is empty. In FIG. 4b a droplet of a precipitant 38 is applied onto the first essentially planar bottom surface section 33a. In FIG. 4c a droplet of a test liquid 37 is applied onto the droplet a precipitant 38. The droplets could alternatively be applied in opposite order.

In FIG. 4d a cover 32 is applied onto the well plate comprising the well and the droplets 37, 38 are pressed slightly down to provide a relatively large contact area between the droplets and the top surface of the cover 32. Thereby analysis of a crystallized structure of the test liquid 37 is simple to perform optically without the need of removing the cover.

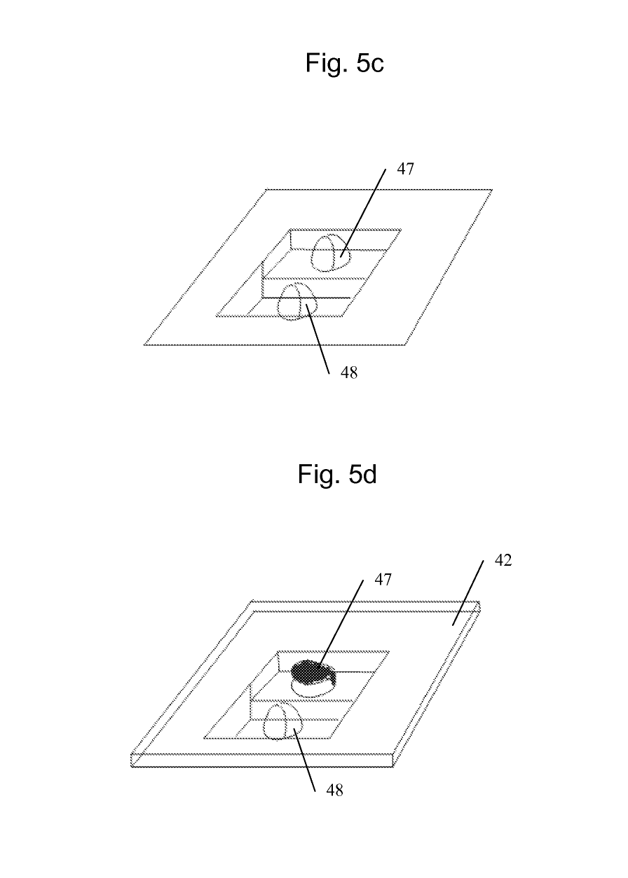

FIGS. 5a-d shows a well of a crystallization system comprising 2 chambers as described above, in 4 different stages of use. The well has a first essentially planar bottom surface section 43a, a second essentially planar bottom surface section 43c and a well border edge 43b. The well border edge 43b has a height D from the first essentially planar bottom surface section, which is preferably as D₁, D₂ and/or D₃ described above and a height D′ from the second essentially planar bottom surface section, which may be larger than D e.g. from about 1.1 times D about 5 times D.

In FIG. 5a the well is empty. In FIG. 5b a droplet of a test liquid 47 is applied onto the first essentially planar bottom surface section 43a. In FIG. 5c a droplet of a precipitant 48 is applied onto the second essentially planar bottom surface section 43c.

In FIG. 5d a cover 42 is applied onto the well plate comprising the well and the droplet of test liquid 47 is pressed slightly down to provide a relatively large contact area between the test liquid droplet 47 and the top surface of the cover 42. Thereby analysis of a crystallized structure of the test liquid 47 is simple to perform optically without the need of removing the cover.

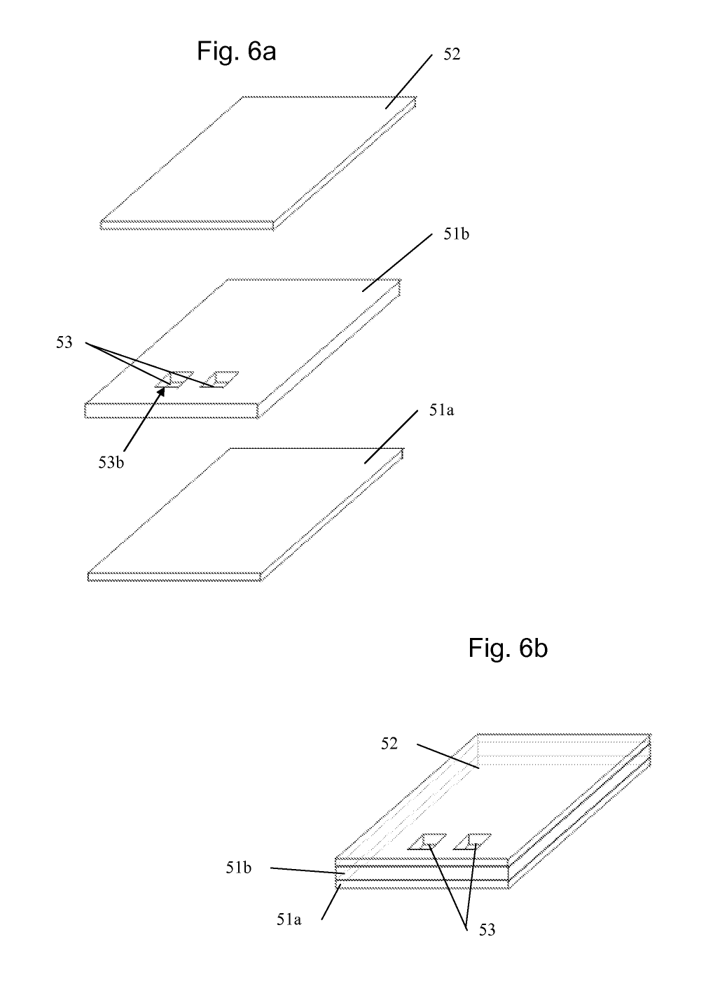

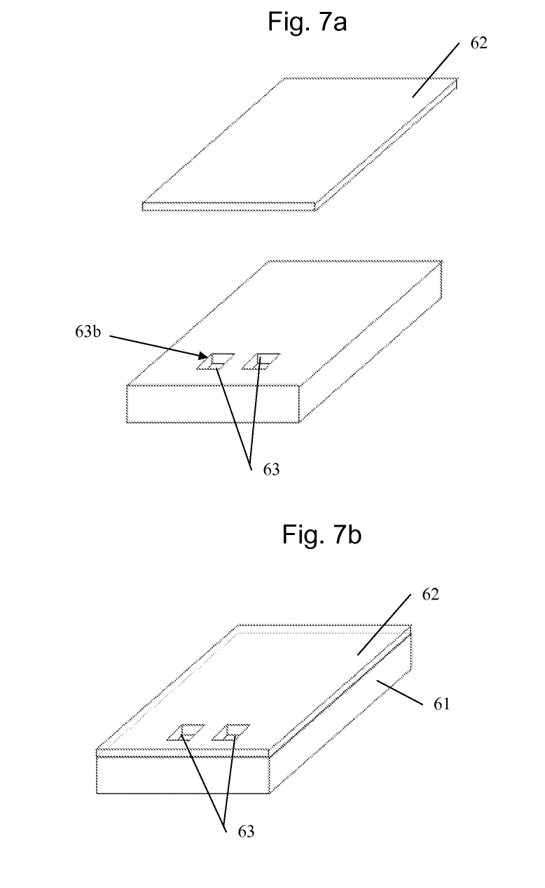

FIGS. 6a and 6b illustrate a fourth crystallization system of the invention which is similar but not identical with the first crystallization device shown in FIGS. 1a and 1b. The fourth crystallization system shown in FIGS. 6a and 6b comprises a well plate 51a, 51b and a cover 52. In FIG. 6a the well plate 51a, 51b is under production and it is seen that it comprises a bottom plate 51a and a perforated plate 51. The perforations 53′ have peripheries which provide the well border edges 53b in the finished well plate 51a, 51b. Furthermore the cover 52 is shown separated from the well plate 51a, 51b. In FIG. 6b the bottom plate 51a and a perforated plate 51b are fixed to each other e.g. by gluing, by heating and/or by mechanical fixation such as a not shown clamp. A plurality of wells 53 will thereby be provided in the well plate 51a, 51b. In case the wells should be of the double type described above the liquid barriers of the wells could e.g. be provided on the surface of the bottom plate 51a which provides the bottom of the respective wells 53 of the well plate 51a, 51b. In FIG. 6b the cover 52 is applied onto the well plate 51a, 51b. As shown at least a part of the cover 52 is of transparent material.

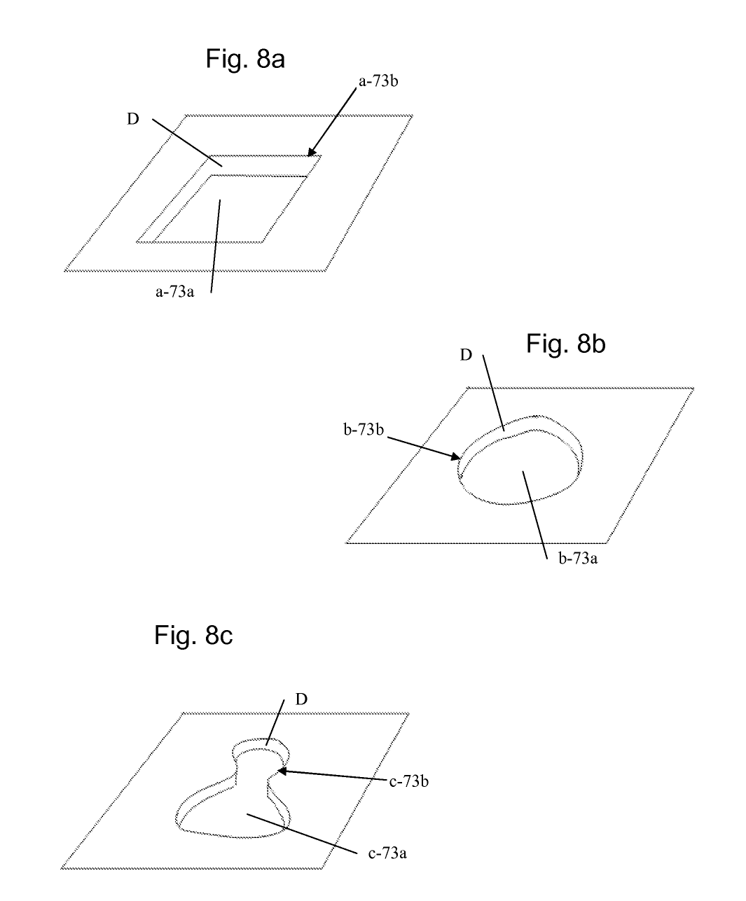

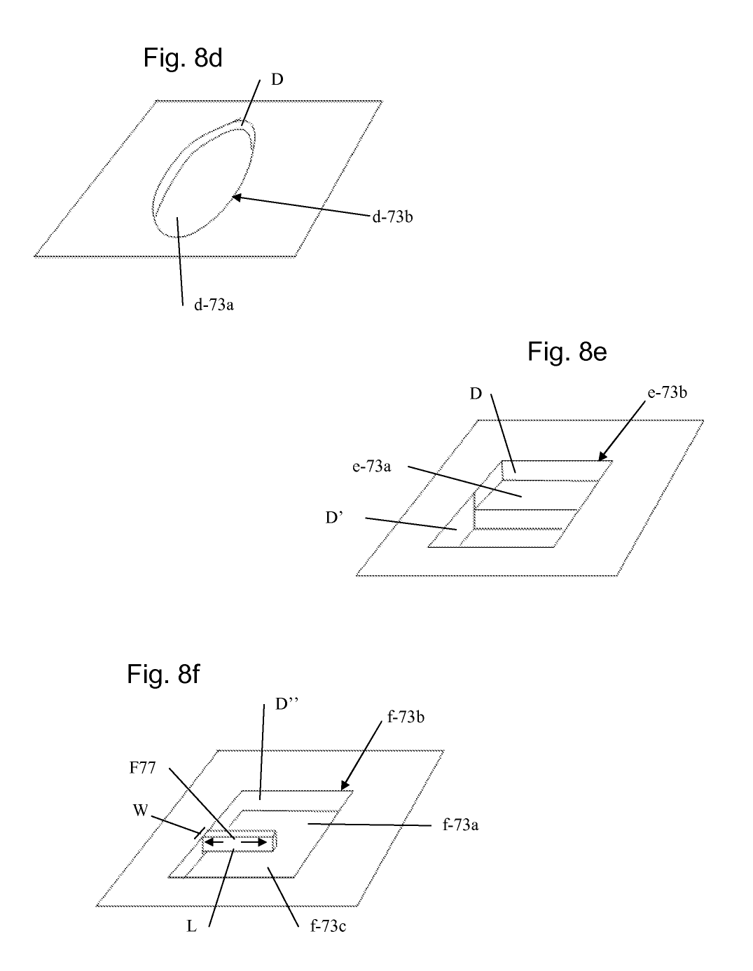

FIGS. 7a and 7b illustrate a fifth crystallization system of the invention which is similar but not identical with the first crystallization device shown in FIGS. 1a and 1b. The fifth crystallization system shown in FIGS. 7a and 7b comprises a well plate 61 and a cover 62 as seen in FIG. 7a. The well plate 61 is provided by one relatively thick plate comprising the wells 63. The wells 63 are for example provided by molding the whole well plate 61, such as by injection molding or by cutting the wells 63 by a laser cutter. In case the wells 63 should be of the double type described above the liquid barriers of the wells 63 could e.g. be provided in the molding step or in the or in a separate cutting step. Furthermore the cover 62 is shown separated from the well plate 61 in FIG. 7a. In FIG. 7b the cover 62 is applied onto the well plate 61. As shown at least a part of the cover 62 is of transparent material.

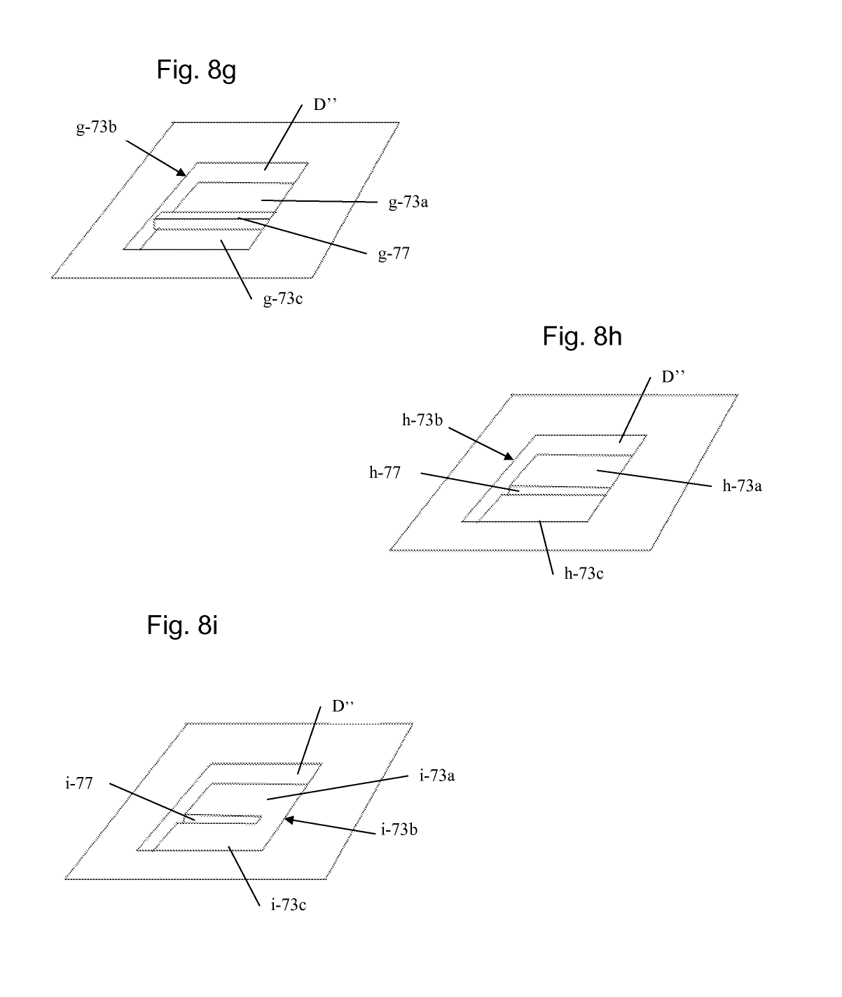

FIGS. 8a-8i are schematic illustrations of 9 different well shapes. The shown wells are examples of wells which could be comprised in the well plate of the crystallization system of the invention.

The well shown in FIG. 8a is similar to the wells 3 shown in FIG. 1a and comprises a bottom surface which constitutes the first essentially planar bottom surface section a-73a, and a well border edge a-73b. The well border edge has a height D from the first essentially planar bottom surface section a-73a, which is preferably as D₁, D₂ and/or D₃ described above. The well shown in FIG. 8a has a shape defined by the well border edge a-73b which is rectangular.

The well shown in FIG. 8b comprises a bottom surface which constitutes the first essentially planar bottom surface section b-73a, and a well border edge b-73b. The well border edge has a height D from the first essentially planar bottom surface section b-73a, which is preferably as D₁, D₂ and/or D₃ described above. The well shown in FIG. 8b has a shape defined by the well border edge b-73b which is round.

The well shown in FIG. 8c comprises a bottom surface which constitutes the first essentially planar bottom surface section c-73a, and a well border edge c-73b. The well border edge has a height D from the first essentially planar bottom surface section c-73a, which is preferably as D₁, D₂ and/or D₃ described above. The well shown in FIG. 8c has a shape defined by the well border edge c-73b which is dumbbell shaped.

The well shown in FIG. 8d comprises a bottom surface which constitutes the first essentially planar bottom surface section d-73a, and a well border edge d-73b. The well border edge has a height D from the first essentially planar bottom surface section d-73a, which is preferably as D₁, D₂ and/or D₃ described above. The well shown in FIG. 8d has a shape defined by the well border edge d-73b which is oval.

The well shown in FIG. 8e is similar to the well shown in FIG. 5a and comprises a first essentially planar bottom surface section e-73a, a second essentially planar bottom surface section e-73c and a well border edge e-73b. The well shown in FIG. 8e has a shape defined by the well border edge e-73b which is rectangular. In the shown well the first essentially planar bottom surface section e-73a and the second essentially planar bottom surface section e-73c have essentially the same size. It should be understood that they may have different sizes. Or that there could be several second essentially planar bottom surface sections. The well border edge e-73b has a height D from the first essentially planar bottom surface section, which is preferably as D₁, D₂ and/or D₃ described above and a height D′ from the second essentially planar bottom surface section, which may be larger than D e.g. from about 1.1 times D about 5 times D or even larger.

The well shown in FIG. 8f comprises a first essentially planar bottom surface section f-73a, a second essentially planar bottom surface section f-73c and a well border edge f-73b. The first essentially planar bottom surface section f-73a and the second essentially planar bottom surface section f-73c are in the same plane (first bottom plane) and are partly separated by an oblong liquid barrier f-77 in the form of a ridge and with a length L and a width W.

The well shown in FIG. 8f has a shape defined by the well border edge f-73b which is rectangular. In the shown well the first essentially planar bottom surface section f-73a and the second essentially planar bottom surface section f-73c have essentially the same size. It should be understood that they may have different sizes. Or that there could be several second essentially planar bottom surface sections. The well border edge f-73b has a height D″ from both the first essentially planar bottom surface section f-73a and the second essentially planar bottom surface section f-73c, which is preferably as D₁, D₂ and/or D₃ described above. In another preferred aspect of the invention the crystallization system when comprising a not shown cover is a double chamber crystallization system where the height D″ preferably is as D₄ described above.

The well shown in FIG. 8g comprises a first essentially planar bottom surface section g-73a, a second essentially planar bottom surface section g-73c and a well border edge g-73b. The first essentially planar bottom surface section g-73a and the second essentially planar bottom surface section g-73c are in the same plane (first bottom plane) and are totally separated by an oblong liquid barrier g-77 in the form of a ridge extending from well border edge g-73b to well border edge g-73b and with a length L and a width W.

The well shown in FIG. 8g has a shape defined by the well border edge g-73b which is rectangular. In the shown well the first essentially planar bottom surface section g-73a and the second essentially planar bottom surface section g-73c have essentially the same size. It should be understood that they may have different sizes. Or that there could be several second essentially planar bottom surface sections. The well border edge g-73b has a height D″ from both the first essentially planar bottom surface section g-73a and the second essentially planar bottom surface section g-73c, which is preferably as D₁, D₂ and/or D₃ described above. In another preferred aspect of the invention the crystallization system when comprising a not shown cover is a double chamber crystallization system where the height D″ preferably is as D₄ described above.

The well shown in FIG. 8h comprises a first essentially planar bottom surface section h-73a, a second essentially planar bottom surface section h-73c and a well border edge h-73b. The first essentially planar bottom surface section h-73a and the second essentially planar bottom surface section h-73c are in the same plane (first bottom plane) and are totally separated by an oblong liquid barrier h-77 in the form of an indentation extending from well border edge h-73b to well border edge h-73b and with a length L and a width W.

The well shown in FIG. 8h has a shape defined by the well border edge h-73b which is rectangular. In the shown well the first essentially planar bottom surface section h-73a and the second essentially planar bottom surface section h-73c have essentially same size. It should be understood that they may have different sizes. Or that there could be several second essentially planar bottom surface sections. The well border edge h-73b has a height D″ from both the first essentially planar bottom surface section h-73a and the second essentially planar bottom surface section h-73c, which is preferably as D₁, D₂ and/or D₃ described above. In another preferred aspect of the invention the crystallization system when comprising a not shown cover is a double chamber crystallization system where the height D″ preferably is as D₄ described above.

The well shown in FIG. 8i comprises a first essentially planar bottom surface section i-73a, a second essentially planar bottom surface section i-73c and a well border edge i-73b. The first essentially planar bottom surface section i-73a and the second essentially planar bottom surface section i-73c are in the same plane (first bottom plane) and are partly separated by an oblong liquid barrier i-77 in the form of an indentation and with a length L and a width W.

The well shown in FIG. 8i has a shape defined by the well border edge i-73b which is rectangular. In the shown well the first essentially planar bottom surface section i-73a and the second essentially planar bottom surface section i-73c have essentially the same size. It should be understood that they may have different sizes. Or that there could be several second essentially planar bottom surface sections. The well border edge i-73b has a height D″ from both the first essentially planar bottom surface section i-73a and the second essentially planar bottom surface section i-73c, which is preferably as D₁, D₂ and/or D₃ described above. In another preferred aspect of the invention the crystallization system when comprising a not shown cover is a double chamber crystallization system where the height D″ preferably is as D₄ described above.

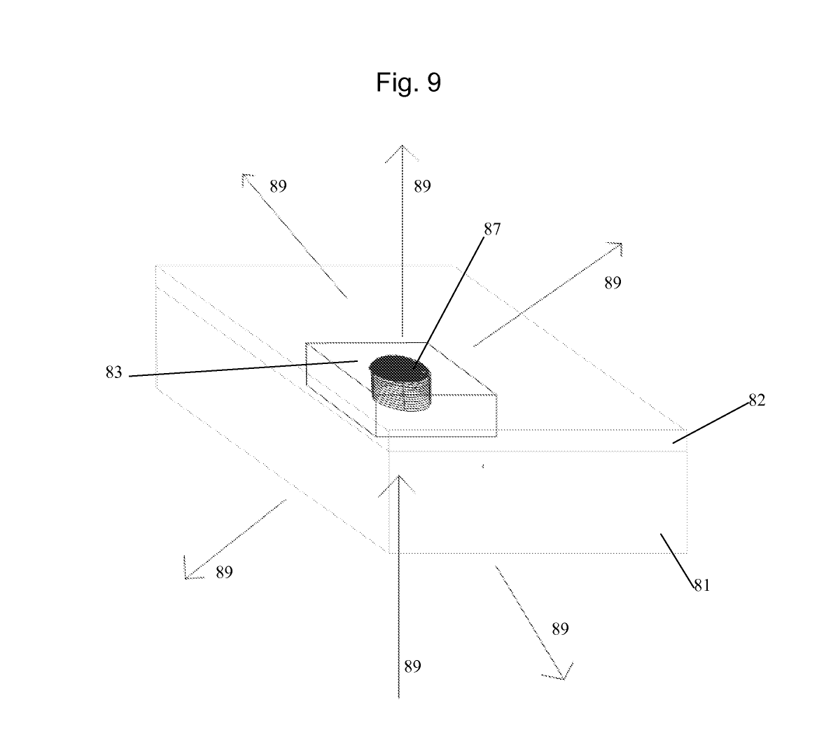

FIG. 9 shows a crystallization system comprising a well plate 81 and a cover 82. The well plate 81 comprises one or more wells 83 where only one is shown. In the well 83 is a target liquid 87 with a crystallized structure of a target molecule which is subjected to an analysis. The arrows 89 indicates that the analysis is performed optically using a source of electro-magnetic radiation illuminates the liquid target sample 87. Read out could for example include diffraction absorption, transmission, fluorescence or luminescence from the drop through interaction with electromagnetic radiation including: X-rays, UV-light, visible light or IR light.

1-56. (canceled)

57. A crystallization system comprising a well plate and a cover for said well plate, at least one of said well plate and said cover comprises at least a transparent window, said well plate comprises at least one well comprising a bottom surface comprising a first essentially planar bottom surface section and a second essentially planar bottom surface section in a first bottom plane and a well border wall provided by a well border edge surrounding said planar bottom surface section, said cover comprises a first essentially planar top surface section in a first top plane adapted to face said first essentially planar bottom surface section, said first and said second essentially planar bottom surface sections are totally or partly separated by a liquid barrier, provided by one or more of a low tension surface barrier, a ridge and an indentation.

58. A crystallization system as claimed in claim 57, wherein said liquid barrier is oblong with a first and a second barrier end, said first and said second barrier end respectively have a shortest barrier-border distance to or coincide with said well border edge, the length of the liquid barrier preferably being at least as long as any shortest barrier-border distance, wherein said shortest barrier-border distance is up to about 10 mm.

59. A crystallization system as claimed in claim 57, wherein said liquid barrier extends from well border edge to well border edge.

60. A crystallization system as claimed in claim 57, wherein said first bottom plane and said first top plane are essentially parallel and have a ‘first bottom plane-cover distance’ D₁ of from about 10 μm to about 1000 μm, when the cover is applied onto said well plate.

61. A crystallization system as claimed in claim 57, wherein at least a section of said bottom wall is transparent to provide at least a transparent window into said well.

62. A crystallization system as claimed in claim 57, wherein said first planar bottom surface section provides a bottom surface of a first chamber and said second planar bottom surface section provides a bottom surface of a second chamber when said cover is applied onto said well border edge, said first and said second chamber being in vapor connection with each other.

63. A crystallization system as claimed in claim 57, wherein said bottom surface is at least about 1 mm².

64. A crystallization system as claimed in claim 57, wherein at least a section of said cover is transparent to provide at least a transparent window into said well, preferably said cover being of a transparent material.

65. A crystallization system as claimed in claim 57, wherein said cover is arranged to provide a tightening between said cover and said well border edge when the cover is applied onto the well plate.

66. A crystallization system as claimed in claim 57, wherein said cover is a shaped cover comprising an outer edge for positioning onto said well plate.

67. A crystallization system as claimed in claim 57, wherein at least a part of at least one of said well and said cover is made from an X-ray transparent material.

68. A crystallization system as claimed in claim 57, wherein the crystallization system comprises a double chamber crystallization system comprising a well plate and a removable cover for said well plate, at least one of said well plate and said cover comprises at least a transparent window, said well plate comprises at least one well comprising a first essentially planar bottom surface section and a second essentially planar bottom surface section, and a well border wall provided by a well border edge surrounding said first and said second essentially planar bottom surface sections, said first essentially planar bottom surface section and said second essentially planar bottom surface section are arranged in a common bottom plane, wherein said first planar bottom surface section provides a bottom surface of a first chamber and said second planar bottom surface section provides a bottom surface of a second chamber when said cover is applied onto said well plate, said first and said second chamber being in vapor communication with each other.

69. A method of producing a crystallization system as claimed in claim 57, said method comprises providing a bottom plate, a perforated plate and a cover, mounting said bottom plate to said perforated plate to provide a well plate comprising a plurality of wells each having a bottom surface.

70. A method as claimed in claim 69, said method comprises providing said bottom plate with a plurality of liquid barriers so that each of said wells is provided to have a first essentially planar bottom surface section and a second essentially planar bottom surface section in a first bottom plane and a well border wall provided by a well border edge surrounding said planar bottom surface section, said liquid barriers are provided by one or more of a low tension surface barrier, a ridge and an indentation.

71. A method as claimed in claim 69, wherein said liquid barrier being provided in said bottom plate prior to mounting it to said perforated plate.

72. A kit comprising a crystallization system and a test liquid, said crystallization system comprising a well plate and a cover for said well plate, at least one of said well plate and said cover comprises at least a transparent window, said well plate comprises at least one well comprising a bottom surface comprising a first essentially planar bottom surface section in a first bottom plane and a well border wall provided by a well border edge surrounding said planar bottom surface section, said cover comprises a first essentially planar top surface section in a first top plane adapted to face said first essentially planar bottom surface section, such that said first bottom plane and said first top plane are essentially parallel and have a first bottom plane—first top plane distance D₃ when the cover is applied onto said well border edge, which first bottom plane—first top plane distance D₃ is smaller than the height of the test liquid when it is applied as a droplet onto said essentially planar bottom surface section, wherein the crystallization system is as claimed in claim 57.

73. A kit as claimed in claim 72, wherein the test liquid is a target molecule solution, the target being a macromolecule.

74. A kit as claimed in claim 72, wherein the test liquid is a target molecule solution, the target being from proteins, nucleic acids, nucleic acids analogues, carbohydrates, lipids more preferably selected from the group of protein of 500 Dalton or more, single and double stranded DNA, RNA, PNA and LNA, and drug candidates.

75. A kit as claimed in claim 72, wherein the test liquid is a target molecule solution comprising at least a dissolved target molecule, a solvent and optionally a precipitant.

76. A kit as claimed claim 72, wherein the test liquid is an aqueous solution further comprising a surfactant.

77. A kit as claimed in claim 72, further comprising a separate precipitant.
